(19) United States
(12) Patent Application Publication
(10) Pub. No.: US 2012/0202894 A1
(43) Pub. Date: Aug. 9, 2012

(54) Rapidly Soluble Solid Pharmaceutical Preparations Containing Amphiphilic Copolymers Based On Polyethers In Combination With Hydrophilic Polymers

(75) Inventors: Karl Kolter, Limburgerhof (DE); Dejan Djuric, Mannheim (DE); Stefan Fischer, Freinsheim (DE)

(73) Assignee: BASF SE, Ludwigshafen (DE)

(21) Appl. No.: 13/496,656

(22) PCT Filed: Sep. 17, 2010
(86) PCT No.: PCT/EP2010/063735

§ 371 Date: Mar. 16, 2012

(30) Foreign Application Priority Data
Sep. 18, 2009 (EP) 09170702.6

(51) Int. Cl. A61K 47/34; A61K 47/38

(52) U.S. Cl. 514/772.2; 514/772.5; 514/772.7

(57) ABSTRACT

Dosage forms comprising formulations of sparingly water-soluble active ingredients in a polymer matrix composed of amphiphilic polyether copolymers and of at least one hydrophilic polymer, in which the sparingly water-soluble active ingredient is present in amorphous form in the polymer matrix.

The present invention relates to solid pharmaceutical formulations comprising sparingly water-soluble active ingredients and amphiphilic copolymers which are obtained by polymerizing vinyl acetate and N-vinyllactams in the presence of a polyether, in combination with hydrophilic polymers which are capable of influencing the stability of the formulation and/or the release of the biologically active substance. The invention further relates to processes for producing these formulations and to the use thereof.

The corresponding copolymers based on polyethers function as solubilizers for the sparingly water-soluble active ingredients.

In the production of homogeneous formulations, especially of biologically active substances, the solubilization of hydrophobic, i.e. sparingly water-soluble, substances has gained very great practical significance.

Solubilization is understood to mean the solubilizing of substances which are sparingly soluble or insoluble in a particular solvent, especially water, by interface-active compounds, the solubilizers. Such solubilizers are capable of converting sparingly water-soluble or water-insoluble substances to clear, at most opalescent, aqueous solutions, without the chemical structure of these substances undergoing any change in the process.

In the solubilizates produced, the sparingly water-soluble or water-insoluble substance is present in colloidally dissolved form in the molecular associates of the surface-active compounds which form in aqueous solution, for example, hydrophobic domains or micelles. The resulting solutions are stable or metastable monophasic systems with a visually clear to opalescent appearance.

In the case of pharmaceutical formulations, the bioavailability and hence the action of medicaments can be enhanced by the use of solubilizers.

A further desirable requirement on solubilizers is the ability to form so-called “solid solutions” with sparingly soluble substances. The term “solid solution” describes a state in which a substance is distributed in colloidal dispersion or ideally molecular dispersion in a solid matrix, for example, a polymer matrix. Such solid solutions lead, for example, when used in solid pharmaceutical administration forms of a sparingly soluble active ingredient, to improved release of the active ingredient. An important requirement on such solid solutions is that they are stable over a long period even in the course of storage, which means that the active ingredient does not crystallize out. Moreover, the capacity of the solid solution or, in other words, the ability to form stable solid solutions with maximum active ingredient contents is also of significance.

WO 2007/051743 discloses the use of amphiphilic water-soluble or water-dispersible copolymers of N-vinyllactam, vinyl acetate and polyethers as solubilizers for pharmaceutical, cosmetic, food technology, agrochemical or other industrial applications. It is described in quite general terms therein that the corresponding graft polymers can also be processed with the active ingredients in the melt.

WO 2009/013202 discloses that such graft polymers of N-vinyllactam, vinyl acetate and polyethers can be melted in an extruder and mixed with pulverulent or liquid active ingredients, the extrusion being described at temperatures significantly below the melting point of the active ingredient.

However, mixing of the molten graft polymer with pulverulent or liquid active ingredients cannot always achieve satisfactorily high and simultaneously stable active ingredient loading. In particular, the achievement of a stable X-ray-amorphous state of the active ingredient is not always possible to a satisfactory degree.

It was an object of the present invention to find improved formulations of sparingly water-soluble active ingredients which, coupled with good bioavailability, enable controlled release and additionally have improved properties with regard to stability toward relatively high air humidity.

Accordingly, formulations of sparingly water-soluble active ingredients in a polymer matrix based on amphiphilic copolymers in combination with hydrophilic polymers have been found.

The amphiphilic copolymers can be obtained by free-radically initiated polymerization of a mixture of
i) 30 to 80% by weight of N-vinyllactam,
ii) 10 to 50% by weight of vinyl acetate, and
iii) 10 to 50% by weight of a polyether,
   with the proviso that the sum of i), ii) and iii) equals 100% by weight.

In one embodiment of the invention, preferred copolymers obtained from:
i) 30 to 70% by weight of N-vinyllactam,
ii) 15 to 35% by weight of vinyl acetate, and
iii) 10 to 35% by weight of a polyether, are used.

Copolymers used with particular preference are obtainable from:
i) 40 to 60% by weight of N-vinyllactam,
ii) 15 to 35% by weight of vinyl acetate, and
iii) 10 to 30% by weight of a polyether.

Copolymers used with very particular preference are obtainable from
i) 50 to 60% by weight of N-vinyllactam,
ii) 25 to 35% by weight of vinyl acetate, and
iii) 10 to 20% by weight of a polyether.

For the preferred and particularly preferred compositions too, the proviso applies that the sum of components i), ii), and iii) equals 100% by weight.

Useful N-vinyllactam includes N-vinylcaprolactam or N-vinylpyrrolidone or mixtures thereof. Preference is given to using N-vinylcaprolactam.

The graft bases used are polyethers. Useful polyethers are preferably polyalkylene glycols. The polyalkylene glycols may have molecular weights of 1000 to 100 000 D [daltons], preferably 1500 to 35 000 D, more preferably 1500 to 10 000 D.

The molecular weights are determined proceeding from the OH number measured to DIN 53240.

Particularly preferred polyalkylene glycols include polyethylene glycols. Also additionally suitable are polypropylene glycols, polytetrahydrofurans or polybutylene glycols, which are obtained from 2-ethyloxirane or 2,3-dimethyloxirane.

Suitable polyethers are also random or block copolymers of polyalkylene glycols obtained from ethylene oxide, propylene oxide and butylene oxides, for example polyethylene glycol-polypropylene glycol block copolymers. The block copolymers may be of the AB type or of the ABA type.

The preferred polyalkylene glycols also include those which are alkylated at one or both OH end groups. Useful alkyl radicals include branched or unbranched C1— to C22-alkyl radicals, preferably C1—C18-alkyl radicals, for example, methyl, ethyl, n-butyl, isobutyl, pentyl, hexyl, octyl, nonyl, decyl, dodecyl, tridecyl or octadecyl radicals.

General processes for preparing the inventive graft copolymers are known per se. They are prepared by free-radically initiated polymerization, preferably in solution, in nonaqueous organic solvents or in mixed nonaqueous/aqueous solvents. Suitable preparation processes are described, for example, in WO 2007/051743 and WO 2009/013202, the disclosure of which is referred to explicitly with regard to the preparation process.

For controlled release, the solubilizing copolymers are used in combination with further polymers which are suitable for influencing the release of the biologically active substance. The degree to which the release is influenced depends generally on the concentration of the release-controlling polymer.

Through the addition of hydrophilic polymers, it is possible to influence the decomposition rate of the resulting extrudates during the release. The increase in the hydrophilicity can achieve more rapid wetting and more rapid decomposition in the release. Suitable for this purpose are especially hydrophilic polymers with low molecular weights (<100 000 daltons). Hydrophilic polymers with higher molecular weight (>100 000 daltons) can be considered as a stabilizer for the resulting solid solution since they increase the rigidity of the matrix and prevent the crystallization of the active ingredient out of oversaturated solutions. As a result, it is possible to prepare stable oversaturated solid solutions which have a particularly high proportion of medicament.

The hydrophilic polymers are generally water-soluble, at least in a particular pH range. In this context, “water-soluble” means that at least 0.1 g dissolves in 1 ml at 20° C.

Examples of suitable hydrophilic polymers include: polyvinylpyrrolidones with K values of 12 to 90, N-vinylpyrrolidone copolymers, for example copolymers with vinyl esters such as vinyl acetate or vinyl propionate, especially copolymers of N-vinylpyrrolidone and vinyl acetate in a weight ratio of 60:40, polyvinyl alcohols, hydroxyalkylated cellulose derivatives such as hydroxypropylcellulose (HPC) or hydroxypropylmethylcellulose (HPMC), hydroxyalkylated and carboxyalkylated cellulose derivatives, acrylic acid-methacrylic acid copolymers.

Additionally suitable are polyethylene glycols with mean molecular weights of 1000 to 6000. Additionally suitable are graft polymers of polyethylene glycol and polyvinyl alcohol units, as commercially available in the form of Kollicoat® IR, from BASF, or mixtures of such graft polymers with polyvinyl alcohol.

In the context of the present invention, the term “water-insoluble” or “sparingly soluble” is used according to DAB 9. According to DAB 9 (Deutsches Arzneimittelbuch [German Pharmacopeae]), the solubility of substances is classified as follows: low solubility (soluble in 30 to 100 parts of solvent); sparingly soluble (soluble in 100 to 1000 parts of solvent); virtually insoluble (soluble in more than 10000 parts of solvent), based in each case on one part of substance to be dissolved at 20° C.

The solid formulations can be produced by methods known per se.

In one embodiment, all ingredients of the formulations are first brought into solution together in a suitable solvent and the solvent is then removed. This can be done by means of all possible kinds of drying processes, for example by means of spray drying, fluidized bed drying, drying using supercritical gases, freeze drying, evaporation.

In a preferred procedure, the solid formulations are produced by extrusion. The polymers can be supplied to the extruder either in pulverulent form or in the form of solutions or dispersions. The dispersions or solutions of the polymer can be converted to solid form by removing the dispersant or solvent in the extruder in the molten state and cooling the melt.

The melt thus obtained can then be cooled and granulated. This is done by so-called hot-cutting or cooling under air or protective gas, for example, on a Teflon or chain belt and subsequent granulation of the cooled melt extrudate. However, cooling is also possible in a solvent in which the polymers do not have significant solubility.

The following methods A-E can be used in principle:

| | |
|---|---|
| A | Physical powder mixing of polymers and active ingredient, and |
| | supply of this powder mixture in the extruder |
| B | Supply of the active ingredient via a separate bypass into the |
| | unmolten polymer mixture |
| C | Supply of the active ingredient via a side feed into molten polymers |
| D | Polymer solution with active ingredient dispersed or dissolved |
| | therein in partly degassed polymer melt or unmelted polymer mixture |
| E | Process according to A-D, except that solvent is additionally |
| | introduced into the extruder and evaporated again |

For the process according to the invention, suitable extruder types in principle are the customary extruder types known to those skilled in the art. Typically, these comprise a housing, a drive unit with transmission, and a process unit which consists of the extruder shaft or shafts equipped with the screw elements, modular construction being assumed in this case.

The extruder consists of a plurality of sections, which are each assigned to particular process units. Each of these sections consists of one or more barrels (barrel blocks) as the smallest independent unit and the corresponding screw sections with the screw elements corresponding to the process task.

The individual barrels should be heatable. In addition, the barrels may also be designed for cooling, for example, for cooling with water. The individual barrel blocks are preferably independently heatable and coolable, such that different temperature zones can also be established along the extrusion direction.

The extruder is advantageously configured as a corotatory twin screw extruder. The screw configuration may have different shear levels according to the product. The screw configuration can be matched to the particular requirements, according to the composition of the formulation, with the customary variable construction elements such as conveying elements, kneading elements, backup elements and the like.

Suitable twin screw extruders may have a screw diameter of 16 to 70 mm and a length of 25 to 40 D.

The entire extruder is formed from barrel blocks, whose temperatures can be controlled individually. The first two barrels may be temperature-controlled for the purpose of better material intake. From the third barrel, a constant temperature is preferably established, which should be selected specifically to the material and depends especially on the melting point of the active ingredient used and the glass transition temperature of the polymer. The resulting product temperature typically, however, depends on the shear level of the screw element used and may in some cases be 20-30° C. higher than the barrel temperature established.

The melting zone may be followed downstream by a venting zone, which is advantageously operated at ambient pressure.

The round dies used may have a diameter of 0.5 to 5 mm. Other die forms such as slot dies may likewise be used, in particular when a greater material throughput is desired.

The resulting extrudates can be processed with a granulator to pellets which can in turn be comminuted (ground) further to a powder. The pellets or powder can be filled into capsules or pressed to tablets using customary tableting assistants. In this context, it is also possible to use further release-controlling assistants.

In addition, it is possible to use, during the extrusion, water, organic solvents, buffer substances or plasticizers. Especially water or volatile alcohols are options for this purpose. This process enables reaction at relatively low temperature. The amounts of solvent or plasticizer are typically between 0 and 30% of the extrudable material. The water or solvent can already be removed by a venting point in the extruder at standard pressure, or by applying reduced pressure. Alternatively, these components evaporate when the extrudate leaves the extruder and the pressure is reduced to standard pressure. In the case of less volatile components, the extrudate can correspondingly be dried subsequently.

In a particular variant of the production process, directly after the extrusion, the thermoplastic material is calendered to a tablet-like compact which constitutes the ultimate administration form. In this variant, it may be advisable to add further constituents, for example polymers for adjusting the glass transition temperature and the melt viscosity, disintegrants, solubilizers, plasticizers, dyes, flavorings, sweeteners, etc. actually before or during the extrusion. In principle, these substances can also be used when the extrudate is first comminuted and then pressed to tablets.

To adjust the glass transition temperature of the formulation, water-soluble polymers with a high glass transition temperature, for example polyvinylpyrrolidone with K values of 17-120, hydroxyalkyl celluloses or hydroxyalkyl starches can be used. Too high a glass transition temperature of the formulation can be lowered by adding plasticizers. Suitable plasticizers for this purpose are in principle all plasticizers which are also used for pharmaceutical coatings, for example triethyl citrate, tributyl citrate, acetyltributyl citrate, triacetin, propylene glycol, polyethylene glycol 400, dibutyl sebacate, glyceryl monostearate, lauric acid, cetyistearyl alcohol.

Moreover, it is also possible additionally to incorporate surfactants which lower the melt viscosity and hence the extrusion temperature into the formulations. These substances can also have a positive influence on the possible crystallization and bring about better wetting of the formulation and more rapid dissolution. Suitable substances are ionic and nonionic surfactants, for example Solutol® HS 15 (macrogol-15 hydroxystearate), Tween® 80, polyoxyethylated fatty acid derivatives such as Cremophor®RH40 (Polyoxyl 40 Hydrogenated Castor Oil, USP), Cremophor EL (Polyoxyl 35 Castor Oil, USP), poloxamers, Docusate sodium or sodium laurylsulfate.

The still plastic mixture is preferably extruded through a die, cooled and comminuted. Suitable comminution methods are in principle all known techniques customary therefor, such as hot or cold cutting.

The extrudate is cut, for example, with rotating blades or with an air jet and then cooled with air or under protective gas.

It is also possible to lay the extrudate as a melt strand on a cooled belt (stainless steel, Teflon, chain belt) and to granulate it after solidification.

Subsequently, the extrudate can optionally be ground. The formulations are obtained as free-flowing water-soluble powders. Preference is given to establishing particle sizes of 20 to 250 μm.

In addition, it is also possible to process the plastic mixture of polymer and active substance by injection molding.

Surprisingly, the inventive formulations have considerably improved stability, i.e. the active ingredient remains in the molecularly disperse or amorphous state in the formulation and does not crystallize out. As a result, the release properties do not change either over time.

The inventive formulations have higher bioavailability of the active ingredient than the formulations without water-soluble polymer.

The formulations obtained by the process according to the invention can in principle be used in all fields in which only sparingly water-soluble or water-insoluble substances are either to be used in aqueous formulations or are to display their action in an aqueous medium.

According to the invention, the term “sparingly water-soluble” also comprises virtually insoluble substances and means that, for a solution of the substance in water at 20° C. at least 30 to 100 g of water is required per g of substance. In the case of virtually insoluble substances, at least 10 000 g of water are required per g of substance.

In the context of the present invention, sparingly-water soluble substances are preferably understood to mean biologically active substances such as active pharmaceutical ingredients for humans and animals, active cosmetic or agrochemical ingredients, or food supplements or active dietetic ingredients.

In addition, useful sparingly soluble substances to be solubilized also include dyes such as inorganic or organic pigments.

According to the invention, useful biologically active substances include, in principle, all solid active ingredients which have a melting point below the decomposition point under extrusion conditions of the copolymers. The copolymers can generally be extruded at temperatures up to 260° C. The lower temperature limit is guided by the composition of the mixtures to be extruded and the sparingly soluble substances to be processed in each case.

The active pharmaceutical ingredients used are water-insoluble substances or substances with low water solubility according to the DAB 9 definition already mentioned.

The active ingredients may come from any indication sector.

Examples here include benzodiazepines, antihypertensives, vitamins, cytostatics—especially taxol, anesthetics, neuroleptics, antidepressives, antivirals, for example anti-HIV drugs, antibiotics, antimycotics, antidementives, fungicides, chemotherapeutics, urologics, thrombocyte aggregation inhibitors, sulfonamides, spasmolytics, hormones, immunoglobulins, sera, thyroid therapeutics, psychopharmaceuticals, Parkinson's drugs and other antihyperkinetics, ophthalmics, neuropathy preparations, calcium metabolism regulators, muscle relaxants, anesthetics, lipid-lowering drugs, liver therapeutics, coronary drugs, cardiac drugs, immunotherapeutics, regulatory peptides and inhibitors thereof, hypnotics, sedatives, gynaecologicals, gout remedies, fibrinolytics, enzyme preparations and transport proteins, enzyme inhibitors, emetics, blood-flow stimulators, diuretics, diagnostics, corticoids, cholinergics, biliary therapeutics, antiasthmatics, bronchodilators, beta-receptor blockers, calcium antagonists, ACE inhibitors, arteriosclerotic drugs, anti-inflammation drugs, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists, slimming drugs.

Among the abovementioned pharmaceutical formulations, particular preference is given to those which are orally administrable formulations.

The content of inventive solubilizer in the pharmaceutical formulation is, depending on the active ingredient, in the range from 1 to 75% by weight, preferably 5 to 60% by weight, more preferably 5 to 50% by weight.

To produce pharmaceutical administration forms, for example, tablets, the extrudates can be admixed with customary pharmaceutical excipients.

These are substances form the class of the fillers, plasticizers, solubilizers, binders, silicates and disintegrants and adsorbents, lubricants, flow agents, dyes, stabilizers such as antioxidants, wetting agents, preservatives, mold release agents, aromas or sweeteners, preferably fillers, plasticizers and solubilizers.

The fillers added may, for example, be inorganic fillers such as oxides of magnesium, aluminum, silicon, titanium carbonate or calcium carbonate, calcium phosphate or magnesium phosphate or organic fillers such as lactose, sucrose, sorbitol, mannitol.

Suitable plasticizers are, for example, triacetin, triethyl citrate, glyceryl monostearate, low molecular weight polyethylene glycols or poloxamers.

Suitable additional solubilizers are interface-active substances with an HLB (Hydrophilic Lipophilic Balance) value greater than 11, for example hydrogenated castor oil ethoxylated with 40 ethylene oxide units (Cremophor® RH 40), castor oil ethoxylated with 35 ethylene oxide units (Cremophor EL), Polysorbate 80, poloxamers or sodium laurylsulfate.

The lubricants used may be stearates of aluminum, calcium, magnesium and tin, and also magnesium silicate, silicones and the like.

The flow agents used may, for example, be talc or colloidal silicon dioxide.

A suitable binder is, for example, microcrystalline cellulose.

The disintegrants may be crosslinked polyvinylpyrrolidone or crosslinked sodium carboxymethyl starch. Stabilizers may be ascorbic acid or tocopherol.

Dyes are, for example, iron oxides, titanium dioxide, triphenylmethane dyes, azo dyes, quinoline dyes, indigotin dyes, carotenoids, in order to dye the administration forms, opacifiers, such as titanium dioxide or talc, in order to increase the transparency and to save dyes.

In addition to use in cosmetics and pharmacy, the formulations produced in accordance with the invention are also suitable for use in the foods sector, for example, for the incorporation of sparingly water-soluble or water-insoluble nutrients, assistants or additives, for example, fat-soluble vitamins or carotenoids. Examples include drinks, colored with carotenoids.

The use of the formulations obtained in accordance with the invention in agrochemistry may include formulations which comprise pesticides, herbicides, fungicides or insecticides, and in particular also those formulations of crop protection compositions which are used as formulations for spraying or watering.

With the aid of the process according to the invention, it is possible to obtain so-called solid solutions comprising sparingly soluble substances. Solid solutions refer in accordance with the invention to systems in which no crystalline components of the sparingly soluble substance are observed.

On visual assessment of the stable solid solutions, no amorphous constituents are evident. The visual assessment can be effected with a light microscope either with or without a polarization filter at 40-fold magnification.

In addition, the formulations can also be examined for crystallinity or amorphicity with the aid of XRD (X-Ray Diffraction) and DSC (Differential Scanning Calorimetry).

The formulations obtained by the process according to the invention are, as stated, present in amorphous form which means that the crystalline components of the biologically active substance are less than 5% by weight. The amorphous state is preferably checked by means of DSC or XRD. Such an amorphous state can also be referred to as an X-ray-amorphous state.

The process according to the invention allows the production of stable formulations with a high active ingredient loading and good stability with regard to the amorphous state of the sparingly soluble substance.

The process according to the invention allows the production of stable formulations with high active ingredient loading.

Surprisingly, such combinations have significantly reduced moisture sensitivity, i.e. the formulations can be stored at high air humidities without the active ingredient crystallizing out.

### EXAMPLES

### Preparation of Polymer 1

In a stirred apparatus, the initial charge without the portion from feed 2 was heated to 77° C. under an N2 atmosphere. When the internal temperature of 77° C. had been attained the portion from feed 2 was added and partly polymerized for 15 min. Subsequently, feed 1 was metered in within 5 h and feed 2 within 2 h. Once all feeds had been metered in, the reaction mixture was polymerized for a further 3 h. After the further polymerization, the solution was adjusted to a solids content of 50% by weight.

Initial charge: 25 g of ethyl acetate
104.0 g of PEG 6000
1.0 g of feed 2

Feed 1: 240 g of vinyl acetate
456 g of vinylcaprolactam
240 g of ethyl acetate

Feed 2: 10.44 g of tert-butyl perpivalate (75% by weight in aliphatics mixture)
67.90 g of ethyl acetate

Subsequently, the solvent was removed by a spray process to obtain a pulverulent product. The K value was 16, measured in 1% by weight solution in ethanol.

The twin screw extruder which was used for the production of the formulations described in the examples which follow had a screw diameter of 16 mm and a length of 40D. The entire extruder was formed from 8 individually temperature-controllable barrel blocks. For the purpose of better material intake, the temperatures of the first two barrels were controlled at 20° C. and at 70° C. respectively. From the third barrel, a constant temperature was established.

The solid solutions produced were examined by means of XRD (X-Ray Diffractometry) and DSC (Differential Scanning Calorimetry) for crystallinity and amorphicity using the following equipment and conditions:

### XRD

Instrument: D 8 Advance diffractometer with 9-tube sample changer (from Bruker/AXS)

Measurement method: θ-θ geometry in reflection

2 theta angle range: 2-80°

Step width: 0.02°

Measurement time per angle step: 4.8s

Divergence slit: Göbel mirror with 0.4 mm inserted aperture

Antiscattering slit: Soller slit

Detector: Sol-X detector

Temperature: Room temperature

Generator setting: 40kV/50mA

### DSC

DSC Q 2000 from TA Instruments

Parameters:

Starting weight approx. 8.5 mg

Heating rate: 20K/min

The active ingredient release was effected using USP apparatus (paddle method) 2, 37° C., 50 rpm (BTWS 600, Pharmatest). The extrudate strands were divided by means of a pelletizer to a length of 3 mm and introduced into hard gelatin capsules. The active ingredient released was detected by UV spectroscopy (Lambda-2, Perkin Elmer).

The solid solutions were analyzed by XRD and by DSC and found to be amorphous. The release of the active ingredient in phosphate buffer pH 6.8 was 27% after 2 h; after 10 h, 82% of the active ingredient originally used had been released.

### Example 1

1200 g of polymer 1, 400 g of Kollidon® VA 64 (copolymer of N-vinylpyrrolidone and vinyl acetate in a weight ratio of 60/40) and 400 g of cinnarizine (melting point 122° C.) were weighed into a Turbula mixing vessel and mixed in a T10 B Turbulu mixer for 10 minutes.

The mixture was extruded under the following conditions:
Zone temperature of 1st barrel: 20° C.; 2nd barrel: 40° C.
Zone temperature from the 3rd barrel: 140° C.
Screw speed 200 rpm
Throughput: 500g/h
Die diameter 3 mm

The solid solutions were examined by XRD and by DSC and were found to be amorphous. After 1 h in 0.1 normal HCl, 95% active ingredient had been released.

### Example 2

1200 g of polymer 1, 400 g of Kollidon VA 64 and 400 g of danazol (melting point 225° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 160° C.
Screw speed 200 rpm
Throughput: 500g/h
Die diameter 3 mm

The solid solutions were examined by XRD and by DSC and were found to be amorphous. After 1 h in 0.1 normal HCl, 95% active ingredient had been released.

### Example 3

1200 g of polymer 1, 500 g of Kollidon VA 64 and 400 g of fenofibrate (melting point 81° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 100° C.
Screw speed 200 rpm
Throughput: 500g/h
Die diameter 3 mm

The solid solutions were examined by XRD and by DSC and were found to be amorphous. After 2 h in 0.1 normal HCl, 100% active ingredient had been released.

### Example 4

1100 g of polymer 1, 400g of Kollidon VA 64, 100 g of PEG 1500 and 400 g of itraconazole (melting point 166° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 130° C.
Screw speed 200 rpm
Throughput: 500g/h
Die diameter 3 mm

The addition of PEG 1500 lowered the extrusion temperature to 130° C., which had no influence on the solid solution. The transparent solid solutions were examined by XRD and by DSC and were found to be amorphous. After 1 h in 0.1 normal HCl, 95% active ingredient had been released.

### Example 5

1200 g of polymer 1, 500g of Kollidon 12 PF (polyvinylpyrrolidone K12) and 300 g of fenofibrate (melting point 81° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 90° C.
Screw speed 200 rpm
Throughput: 500g/h
Die diameter 3 mm

The solid solutions were examined by XRD and by DSC and were found to be amorphous. The addition of Kollidon 12PF achieved more rapid dissolution of the pellets during the release. After 1 h in 0.1 normal HCl, 100% active ingredient had been released.

### Example 6

1200 g of polymer 1, 400g of Kollidon 17 PF (polyvinylpyrrolidone K17) and 400 g of clotrimazole (melting point 148° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 120° C.
Screw speed 200 rpm
Throughput: 500g/h
Die diameter 3 mm 25

The solid solutions were examined by XRD and by DSC and were found to be amorphous. The addition of Kollidon 17PF achieved more rapid dissolution of the pellets during the release. After 1 h in 0.1 normal HCl, 80% active ingredient had been released.

### Example 7

800 g of polymer 1, 800 g of Kollidon 90 (polyvinylpyrrolidone K90) and 400 g of naproxen (melting point 157° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 160° C.
Screw speed 200 rpm
Throughput: 600g/h
Die diameter 3 mm

The solid solutions were examined by XRD and by DSC and were found to be amorphous. After 1 h in 0.1 normal HCl, 89% active ingredient had been released.

### Example 8

1200 g of polymer 1, 400g of Kollicoat® IR (graft polymer of PEG 6000/polyvinyl alcohol), 100 g of PEG 1500 and 400 g of itraconazole (melting point 166° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 140° C.
Screw speed 200 rpm
Throughput: 800g/h
Die diameter 3 mm

The solid solutions were examined by XRD and by DSC and were found to be amorphous. After 1 h in 0.1 normal HCl, 99% active ingredient had been released.

### Example 9

1000 g of polymer 1, 600g of Kollicoat IR and 400 g of fenofibrate (melting point 81° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 130° C.
Screw speed 200 rpm
Throughput: 1000g/h
Die diameter 3 mm

The solid solutions were examined by XRD and by DSC and were found to be amorphous. The release of the active ingredient in 0.1 normal HCl after 2 h was less than 20%. After changing the buffer to pH 6.8 for a further 10 h, a total of 80% active ingredient had been released.

### Example 10

1200 g of polymer 1, 400 g of HPMC and 400g of cinnarizine (melting point 122° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 160° C.
Screw speed 100 rpm
Throughput: 800 g/h
Die diameter 3 mm

The HPMC addition led to more flexible extrudate strands which were easier to granulate. The solid solutions were examined by XRD and by DSC and were found to be amorphous. The release of the active ingredient in 0.1 normal HCl after 2 h was less than 10%; after changing the buffer to pH 6.8, 100% was released.

### Example 11

1000 g of polymer 1, 600 g of HPC and 400 g of carbamazepine (melting point 192° C.) were weighed into a Turbula mixing vessel and mixed in a T1OB Turbula mixer for 10 minutes.

The mixture was extruded under the following conditions:
Zone temperature of 1st barrel: 20° C.; 2nd barrel: 40° C.
Zone temperature from the 3rd barrel: 170° C.
Screw speed 200 rpm
Throughput 600 g/h
Die diameter 3 mm

The solid solutions were examined by XRD and by DSC and were found to be amorphous. After 1 h in 0.1 normal HCl, 95% active ingredient had been released.

1. A dosage form comprising formulations of sparingly water-soluble active ingredients in amorphous form in a polymer matrix comprising amphiphilic polyether copolymers and at least one hydrophilic polymer.

2. The dosage form of claim 1, wherein the polyether copolymers are obtained by free-radically initiated polymerization of a mixture of 30 to 80% by weight of N-vinyllactam,_{—}10 to 50% by weight of vinyl acetate and 10 to 50% by weight of a polyether.

3. The dosage form of claim 1, wherein at least one hydrophilic polymer has a solubility in water at 20° C. of at least 0.1 g/ml.

4. The dosage form of claim 1, wherein the at least one hydrophilic polymer is selected from the group consisting of polyvinylpyrrolidones, N-vinylpyrrolidone copolymers, polyvinyl alcohols, hydroxyalkylated cellulose derivatives, carboxyalkylated cellulose derivatives, acrylic acid-methacrylic acid copolymers, polyethylene glycols, graft polymers of polyethylene glycol and polyvinyl alcohol units, or mixtures of such polymers.

5. The dosage form of claim 1, wherein at least one hydrophilic polymer comprises polyvinylpyrrolidone or copolymers of N-vinylpyrrolidone and vinyl acetate.

6. The dosage form of claim 1, wherein the at least one hydrophilic polymer comprises hydroxypropylcellulose or hydroxypropylmethylcellulose.

7. The dosage form of claim wherein the at least one hydrophilic polymer comprises a graft polymer of polyether and polyvinyl alcohol units.

8. The dosage form of claim 1, wherein the ratio of the polyether copolymer to the hydrophilic polymer is between 1:99 and 99:1.

9. The dosage form of claim 2, wherein the polyether copolymers are obtained from:
i) 40 to 60% by weight of N-vinyllactam
ii) 15 to 35% by weight of vinyl acetate and
iii) 10 to 30% by weight of a polyether.

10. The dosage form of claim 2, wherein the polyether copolymers are obtained from:
i) 50 to 60% by weight of N-vinyllactam
ii) 25 to 35% by weight of vinyl acetate and
iii) 10 to 20% by weight of a polyether.

11. The dosage form of claim 2, wherein the N-vinyllactam comprises N-vinylcaprolactam, N-vinylpyrrolidone or mixtures thereof.

12. The dosage form of claim 1, wherein at least one hydrophilic polymer has a molecular weight below 100,000 Daltons.

13. The dosage form of claim 1, wherein at least one hydrophilic polymer has a molecular weight above 100,000 Daltons.

14. The dosage form of claim 5, wherein at least one hydrophilic polymer comprises a copolymer of N-vinylpyrrolidone and vinyl acetate in a weight ratio of 60:40.

15. The dosage form of claim 5, wherein at least one hydrophilic polymer comprises a polyvinylpyrrolidone with a K value in the range of 12 to 90.
